(19) United States
(12) Patent Application Publication
(10) Pub. No.: US 2023/0372288 A1
(43) Pub. Date: Nov. 23, 2023

(54) Application of Aristolochic Acid IVa in Preparation of Antihistamine or Drug for Treating Pneumonia

(71) Applicant: Institute of Chinese Materia Medica, China Academy of Chinese Medical Sciences, Beijing (CN)

(72) Inventors: Aihua Liang, Beijing (CN); Lianmei Wang, Beijing (CN)

(21) Appl. No.: 18/226,226

(22) Filed: Jul. 25, 2023

Related U.S. Application Data
(63) Continuation of application No. PCT/CN2022/111333, filed on Aug. 10, 2022.

(30) Foreign Application Priority Data
Apr. 12, 2022 (CN) 202210379030.2

(51) Int. Cl. A61K 31/36; A61P 37/08; A61P 11/00

(52) U.S. Cl. CPC A61K 31/36; A61P 37/08; A61P 11/00

(57) ABSTRACT

The present application discloses an application of an aristolochic acid IVa in preparation of an antihistamine or a drug for treating pneumonia, and belongs to the technical field of new medicine uses. It uses the aristolochic acid IVa and a pharmaceutically acceptable salt thereof as active components, the antihistamine effect may be achieved by inhibiting the vascular permeability on the one hand, and the pneumonia treatment may be achieved by reducing the amount of tissue inflammation exudation on the other hand. Compared with existing technologies, the active ingredient aristolochic acid IVa of the present application does not have apparent toxicity and is quite safe, so a new direction for an application of a traditional Chinese medicine containing the aristolochic acid is provided.

### CROSS-REFERENCE TO RELATED APPLICATIONS

The present application is a continuation application of PCT application No. PCT/CN2022/111333 filed on Aug. 10, 2022, which claims the benefit of Chinese Patent Application No. 202210379030.2 filed on Apr. 12, 2022. The contents of all of the aforementioned applications are incorporated by reference herein in their entirety.

### TECHNICAL FIELD

The present application belongs to the technical field of new medicine uses, and specifically relates to an application of an aristolochic acid IVa in preparation of an antihistamine or a drug for treating pneumonia.

### BACKGROUND

Histamine is an allergic and inflammatory medium. Many tissues, especially mast cells of skins, lungs and intestinal mucosae, contain a large number of the histamines. When the tissue is damaged or inflammation and allergic reactions occur, the histamines may be released. The histamine has a strong vasodilator effect, and may increase the permeability of capillary and venule walls, thus plasma leaks into the tissue, leading to local tissue edema. Many diseases are clinically related to histamine release, such as skin allergy, rhinitis, asthma, and allergic shock.

Pneumonia is caused by various pathogens (such as viruses, bacteria, fungi, and parasites), radiation, chemicals, allergies, and other factors. Typical pneumonia is lobar pneumonia or bronchopneumonia caused by common bacteria such as Streptococcus pneumoniae. Atypical pneumonia is pneumonia caused by mycoplasma, chlamydia, legionella, rickettsia, viruses and other unknown microorganisms, herein the atypical pneumonia caused by Severe Acute Respiratory Syndrome (SARS) viruses and Middle East Respiratory Syndrome (MERS) viruses may cause severe pulmonary edema and multiple-organ failure. Virus proliferation may occur in lung organs of a small number of severe or critical patients (about 20%) with novel coronaviruses, to cause damage to alveolar epithelial cells, and then cause the pneumonia, even immune cascade reactions and inflammatory storms, eventually leading to severe atypical bilateral interstitial pneumonia.

The main pathological change of a lung in acute pneumonia is acute inflammatory exudation, and it may cause the pulmonary edema in severe cases. The acute pulmonary edema is an important cause of death in patients with acute lung infection and chemical lung injury (chlorine or phosgene poisoning).

Aristolochic acids are a nitrophenanthrene carboxylic acid compound, and the aristolochic acids are previously believed to have nephrotoxicity and carcinogenicity. But there are many types of the aristolochic acids, and the toxicities of the different aristolochic acids vary greatly. The toxic aristolochic acids are mainly AA-I and AA-II. Some researches show that the aristolochic acid IVa (AA-Iva) does not have apparent toxicity, therefore it is quite safe, and it has a good prospect for medicinal uses. After being retrieved, relevant experimental researches are not found yet on the preparation of antihistamines or drugs for treating pneumonia using the aristolochic acid IVa.

### SUMMARY

A purpose of the present application is to provide a new medicine use for an aristolochic acid IVa, and specifically provide an application of an aristolochic acid IVa and a pharmaceutically acceptable salt thereof as active components in preparation of an antihistamine or a drug for treating pneumonia.

In order to achieve the above purpose, the present application adopts the following technical schemes.

An application of an aristolochic acid IVa in preparation of an antihistamine or a drug for treating pneumonia is provided.

Preferably, the molecular formula of the aristolochic acid IVa is: C₁₇H₁₁NO₈, and the chemical structure formula is:

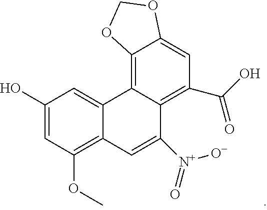 [SMILES: COc1cc(O)cc2c1cc([N+](=O)[O-])c1c(C(=O)O)cc3c(c12)OCO3]

Preferably, the use of the antihistamine includes a use for skin allergy, eczema, dermatitis, rhinitis, or asthma.

Preferably, the pneumonia includes pneumonia and atypical pneumonia caused by pathogens, radiation, chemicals, and allergic factors.

Further preferably, the pathogen includes at least one of viruses, bacteria, fungi, and parasites.

Further preferably, the atypical pneumonia is caused by at least one of SARS viruses, MERS viruses, novel coronaviruses, adenoviruses, influenza viruses and legionella.

Preferably, the drug includes the active ingredient aristolochic acid IVa and a pharmaceutically acceptable carrier.

Preferably, the carrier includes at least one of a diluent, an adhesive, an absorbent, a disintegrant, a dispersant, a wetting agent, a cosolvent, a buffering agent, and a surfactant.

Preferably, the administration mode of the drug is: at least one of oral, sublingual, oral mucosal, intravenous, intramuscular, intraperitoneal, subcutaneous, percutaneous, nasal, and rectal pathways.

Preferably, the drug is in the form of solid, liquid, or gas.

Further preferably, the solid form is powder, tablet, granule, pill, hard capsule or soft capsule, cream, ointment, emplastrum, gel, paste, pulvis or patch.

Further preferably, the liquid form is solution, suspension, injection, syrup, liniment, emulsion, tincture, or elixir.

Further preferably, the gas form is: aerosol or spray.

Another purpose of the present application is to provide an antihistamine, containing an active ingredient aristolochic acid IVa or a medically acceptable salt thereof.

Another purpose of the present application is to provide a drug for treating pneumonia, containing an active ingredient aristolochic acid IVa or a medically acceptable salt thereof.

The present application has the following beneficial effects.

The aristolochic acid IVa of the present application may significantly inhibit the increase in vascular permeability induced by histamines, and show the significant antihistamine effect. At the same time, the aristolochic acid IVa may significantly reduce the amount of inflammation exudation in lung tissues.

From the above experiment, it may be seen that the aristolochic acid IVa of the present application achieves the antihistamine effect by inhibiting the vascular permeability, and at the same time, the aristolochic acid IVa achieves the pneumonia treatment by reducing the amount of tissue inflammation exudation.

Traditional Chinese medicines containing the aristolochic acid have nephrotoxicity. Compared with existing technologies, the active ingredient aristolochic acid IVa of the present application does not have apparent toxicity and is good in safety, so a new direction for an application of the traditional Chinese medicine containing the aristolochic acid is provided.

### BRIEF DESCRIPTION OF THE DRAWINGS

FIG. 1 shows that AA-IVa reduces a histamine-induced increase in vascular permeability (manifested as ear blue staining).

FIG. 2 shows that AA-IVa reduces inflammatory exudation in a lung tissue (hematoxylin-eosin (HE) pathological image).

FIG. 3 shows effects of AA-I and AA-II on the histamine-induced increase in the vascular permeability.

FIG. 4 shows effects of AA-I and AA-II on the inflammatory exudation in the lung tissue (HE pathological image).

FIG. 5 shows comparison of nephrotoxicity among AA-I, AA-II, and AA-IVa (renal pathological image).

Herein, A is a control group, B is a histamine group, C is a histamine+AA-IVa/low-dose group, D is a histamine+AA-IVa/high-dose group, E is a histamine+AA-I group, and F is a histamine+AA-II group.

### DETAILED DESCRIPTION OF THE EMBODIMENTS

In order to make purposes, technical schemes, and advantages of the present application clearer, the technical schemes of the present application are further described in detail below in combination with specific embodiments. Unless otherwise defined, all technical and scientific terms used in this article have the same meaning as commonly understood by those of ordinary skill in the art to which the present application belongs.

### Embodiment 1: Effect of AA-IVa on Histamine-Induced Increase in Mouse Vascular Permeability

1. Test Reagent

Evans Blue (EB): Sinopharm Chemical Reagent Co., Ltd., batch number: 20180125; 0.9% sodium chloride injection: Cisen Pharmaceutical Co., Ltd., batch number: 1801022725; formamide: Tianjin Damao Chemical Reagent Factory, batch number: 20180416; histamine: Sinopharm Chemical Reagent Co., Ltd., batch number: 20160711; and AA-IVa (molecular formula: C₁₇H₁₁NO₈, and molecular weight: 293.27), derived from Chinese traditional herb asarum, purchased from Beijing Saibaicao Technology Co., Ltd., batch number: SH18121005.

2. Test Material

Mouse strain: Institute of Cancer Research (ICR); and gender: male.

Animal weight: 23-25 g of an animal is selected, and a test substance is administered after 1 day of adaptive feeding. Source: Beijing Vital River Laboratory Animal Technology Co., Ltd. Animal requirement: special pathogen free.

Animal feeding location during the experiment: the Barrier Environment Animal Laboratory of the Institute of Chinese Materia Meica China Academy of Chinese Medical Sciences is used.

Feeding condition: a barrier system, 20-26° C. of a temperature, 40-70% of a relative humidity, and all fresh air. Artificial illumination is adopted, and there is a 12-hour light and dark cycle. The animals are fed in a polycarbonate mouse feeding cage, and there are 5 mice per cage.

Feed: a standard mouse pellet feed is used, and provided by Beijing Keao Xieli Feed Co., Ltd.; Drinking water: drinking purified water is used, and a high-pressure sterilized drinking water bottle is replaced twice a week.

3. Test Instrument

Animal balance: Sartorius, Germany, model: BSA3202s-CW and BSA224S-CW; and

Microplate reader: Thermo, USA, model: VARIOSKAN LUX.

4. Test Method

4.1 Animal Grouping

ICR mice are divided into 6 groups, including a negative control group (control): physiological saline is administered by gavage for 3 days, and on the 4th day, the physiological saline is administered by tail vein injection; a histamine model group (histamine): the physiological saline is administered by the gavage for 3 days, and 5 mg/kg of the histamine is administered by the tail vein injection on the 4th day; an AA-IVa low-dose treatment group (histamine+AA-IVa/L): 5 mg/kg of AA-IVa is administered by the gavage for 3 days, and 5 mg/kg of the histamine is administered by the tail vein injection on the 4th day; and an AA-IVa high-dose treatment group (histamine+AA-IVa/H): 10 mg/kg of AA-IVa is administered by the gavage for 3 days, and 5 mg/kg of the histamine is administered by the tail vein injection on the 4th day.

4.2 Drug Preparation

AA-Iva: 0.5 wt % CMC-Na is used for preparation, and the concentration is 0.5 mg/ml and 0.25 mg/ml respectively. Histamine: 0.5 mg/ml of histamine solution is prepared with the physiological saline.

4.3 Modeling Method
(1) Principle: the histamine may cause an increase in permeability of capillary and venule walls, and plasma leaks into tissues, leading to local tissue edema. EB is a commonly used indicator for detecting the vascular permeability. After intravenous injection of mixed solution of histamine and EB into the mice, the increase in the vascular permeability caused by the histamine may be observed in a mouse auricle (the auricle is stained blue).
(2) Method for establishing model of increased auricular vascular permeability induced by histamine: after the histamine (0.5 mg/ml) is mixed with 0.8% EB solution at a ratio of 1:1 (volume), the tail vein injection is performed at 20 ml/kg of a volume according to the weight of each mouse. The auricular blue staining condition of the mouse is observed within 30 min after the histamine is administered, and a picture of the auricular blue staining is taken.
(3) Auricular vascular permeability test (EB exudation test): after the above auricular blue staining is observed and photographed, both ears are cut, and cut into pieces. After being soaked in 2 ml of formamide at a room temperature for 48 h, it is filtered with a 200-mesh stainless steel sieve. A filtrate is taken, and the absorbance (A) is measured at 610 nm. According to an EB standard curve, the amount (μg) of EB dye exudation is calculated.

EB exudation reduction rate (%)=(average value of EB exudation in histamine group−average value of EB exudation in administration group)/average value of EB exudation in histamine group×100%.

5. Statistical Method

Statistical analysis is performed by using SPSS 20.0 software. The variance analysis is performed on the amount of EB exudation from the auricle tissue by using an ANOVA program. Data is expressed as mean±standard deviation (x̅±S), the histamine model group is compared with the control group, and each administration group is compared with the histamine model group, P<0.05 is considered as a significant difference.

6. Test Result

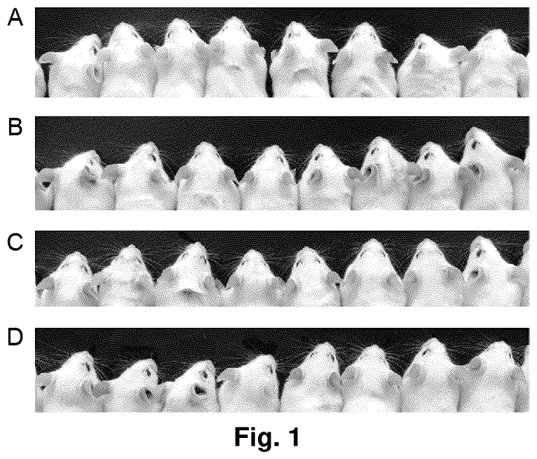

The histamine is used as the model group, and after the histamine is administered, the mouse auricle shows a significant increase in the vascular permeability and apparent blue staining (shown in B of FIG. 1).

Compared with the histamine model group, the ear blue staining of the animals in the different doses of the AA-IVa group is significantly reduced (shown in C of FIG. 1 and D of FIG. 1), and the amount of EB exudation is significantly reduced (P<0.001), it is indicated that AA-IVa has the significant antihistamine effect.

The ears of each mouse in each group are soaked in formamide solution, EB is dissolved out, and then it is measured by using the microplate reader at 610 nm. The amount of EB exuded from the ears of the mouse may be obtained, so the degree of the vascular permeability increased is reflected. Results are shown in Table 1, the amount of EB exudation in the histamine model group is significantly increased compared to the control group (P<0.001 compared to the control group); and the amounts of EB exudation in the different doses of the AA-IVa groups are all significantly decreased compared to the histamine model group (P<0.001 compared to the histamine model group). It is indicated from the results that AA-Iva may significantly inhibit the increase in the vascular permeability induced by the histamine, namely it has the significant antihistamine effect.

TABLE 1
Histamine-induced ear EB exudation alleviated
by different doses of AA-IVa (n = 10)

| | | | Amount | |
|---|---|---|---|---|
| | Drug | Histamine | of EB | Reduction |
| | dose | dose | exudation | rate |
| Group | (mg/kg) | (mg/kg) | (μg) | (%) |
| Control | — | — | 0.31 ± 0.10 | — |
| Histamine | — | 5 | 1.47 ± 0.33*** | — |
| model | | | | |
| AA-IVa/L | 5 | 5 | 0.93 ± 0.20^{###} | 36.7 |
| AA-IVa/H | 10 | 5 | 0.82 ± 0.25^{###} | 44.2 |

Note:
* represents comparison with the control group,
***represents P < 0.001; and
# represents comparison with the histamine model group,
^{###}represents P < 0.001.

### Contrast Example 1: Effects of Aristolochic Acid I (AA-I) and Aristolochic Acid II (AA-II) on Histamine-Induced Mouse Vascular Permeability

1. Test Reagent

EB: Sinopharm Chemical Reagent Co., Ltd., batch number: 20180125; 0.9% sodium chloride injection: Cisen Pharmaceutical Co., Ltd., batch number: 1801022725; formamide: Tianjin Damao Chemical Reagent Factory, batch number: 20180416; histamine: China National Pharmaceutical Group Chemical Reagent Co., Ltd., batch number: 20160711; and AA-I (molecular formula: C₁₇H₁₁NO₇, and molecular weight: 341.27) and AA-II (molecular formula: C₁₆H₉NO₆, Chemical Abstracts Service (CAS) Number: 475-80-9, and molecular weight: 311.25), purchased from Beijing Saibaicao Technology Co., Ltd.

2. Test Material

Same as Embodiment 1.

3. Test Instrument

Same as Embodiment 1.

4. Test Method

4.1 Animal Grouping

ICR mice are divided into 4 groups, including a negative control group (control) (1): physiological saline is administered by gavage for 3 days, and on the 4th day, the physiological saline is administered by tail vein injection; a histamine model group (histamine) (2): the physiological saline is administered by the gavage for 3 days, and on the 4th day, 5 mg/kg of the histamine is administered by the tail vein injection; an AA-I treatment group (histamine+AA-I) (3): 10 mg/kg of AA-I is administered by the gavage for 3 days, and on the 4th day, 5 mg/kg of the histamine is administered by the tail vein injection; and an AA-II treatment group (histamine+AA-II) (4): 10 mg/kg of AA-II is administered by the gavage for 3 days, and on the 4th day, 5 mg/kg of the histamine is administered by the tail vein injection.

4.2 Drug Preparation

Preparation of AA-I and AA-II: 0.5 wt % CMC-Na is used to prepare AA-I and AA-II into 0.5 mg/ml respectively.

Histamine preparation: 0.5 mg/ml of histamine solution is prepared by using the physiological saline.

4.3 Modeling Method

Same as Embodiment 1.

5. Statistical Method

Same as Embodiment 1.

6. Test Result

The histamine is used as the model group, and after the histamine is administered, the mouse auricle shows a significant increase in the vascular permeability and apparent blue staining (shown in B of FIG. 3).

There is no significant difference in ear blue staining between the AA-I and AA-II groups and the histamine model group (shown in E and F of FIG. 3), it is indicated that AA-I and AA-II do not have the significant antihistamine effect.

The ears of each mouse in each group are soaked in formamide solution, EB is dissolved out, and then it is measured by using the microplate reader at 610 nm. The amount of EB exuded from the ears of the mouse may be obtained, so the degree of the vascular permeability increased is reflected. Results are shown in Table 2, the amount of EB exudation in the histamine model group is significantly increased compared to the control group (P<0.001); and there is no significant difference between the AA-I and AA-II groups and the histamine model group, it is indicated that AA-I and AA-II do not have the significant antihistamine effect.

TABLE 2
No significant improvement in histamine-induced
ear EB exudation using AA-I and AA-II (n = 10)

| | | | Amount | |
|---|---|---|---|---|
| | Drug | Histamine | of EB | Reduction |
| | dose | dose | exudation | rate |
| Group | (mg/kg) | (mg/kg) | (μg) | (%) |
| Physiological | — | — | 0.34 ± 0.13 | — |
| saline | | | | |
| Histamine | — | 5 | 1.18 ± 0.26*** | — |
| AA-I | 10 | 5 | 1.01 ± 0.28*** | 14.4% |
| AA- II | 10 | 5 | 1.09 ± 0.34*** | 7.6% |

Note:
(1) * represents comparison with the control group, and
***represents P < 0.001.
(2) There is no statistically significant difference between the AA-I and AA-II groups and the histamine model group.

### Embodiment 2: Histamine-Induced Pneumonia Alleviated by AA-IVa

1. Experimental Method
(1) Model establishment, animal grouping and administration are the same as Embodiment 1.
(2) Pathological examination of lung tissue: after the mouse is euthanized due to vertebral detachment, it is dissected, and the lung tissue is taken out. The lung tissue is fixed with 10% neutral formalin for standby. After being fully fixed with formaldehyde, the submitted tissue is dehydrated step by step with ethanol, transparent with xylene, embedded with paraffin, and prepared conventionally into 3 μm of a paraffin section. HE, Masson, and PAS staining are used respectively, and the tissue inflammation exudation conditions are examined under an optical microscope (DP71 type, OLYMPUS, magnified by 400 times).

2. Experimental Result

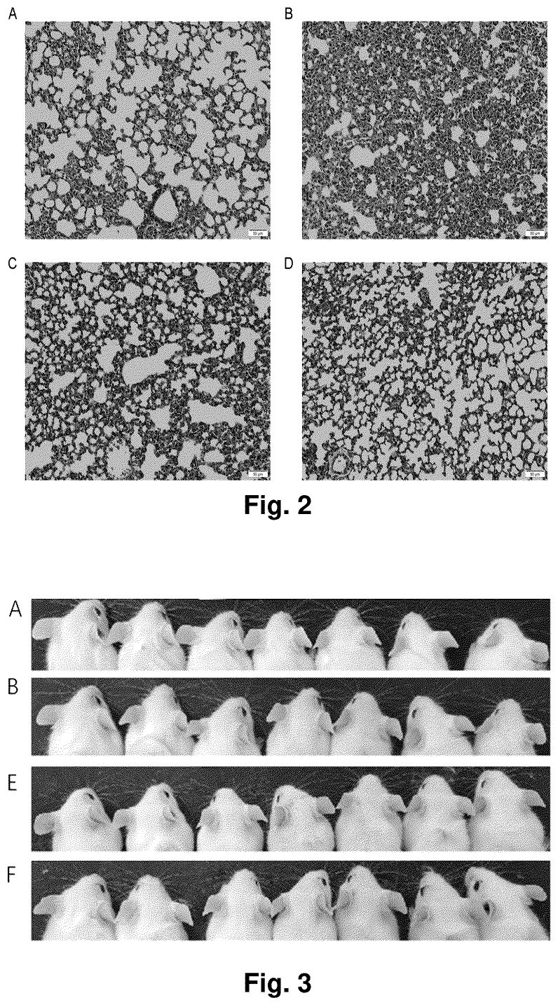

The pathological results of the lung tissue show that: A of FIG. 2 is a normal control group, and shows normal alveolar and tracheal structures in the lung tissue. B of FIG. 2 is a model control group, and shows thickening of alveolar septa and increased exudation of inflammatory cells. C of FIG. 2 is a histamine+AA-IVa/L group, and shows the thickening of alveolar septa and the increased exudation of inflammatory cells, but there is a significant reduction compared to the model group. D of FIG. 2 is a histamine+AA-IVa/H group, and shows the thickening of alveolar septa and the increased exudation of inflammatory cells, but there is a significant reduction compared to the model group.

### Contrast Example 2: Effects of AA-I and AA-II on Histamine-Induced Pneumonia

1. Experimental Method
(1) Model establishment, animal grouping and administration are the same as Contrast example 1.
(2) Pathological examination of lung tissue: same as Embodiment 2.

2. Experimental Result

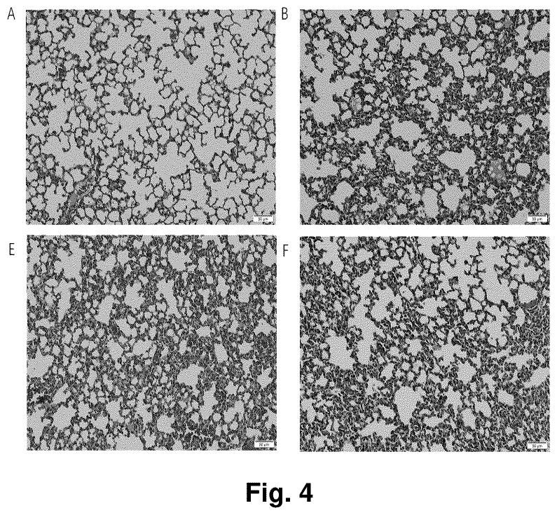

Pathological results of the lung tissue in FIG. 4 show that: A of FIG. 4 is a normal control group, and shows normal alveolar and tracheal structures in the lung tissue. B of FIG. 4 is a histamine model group, and shows thickening of alveolar septa and increased exudation of inflammatory cells, it is indicated that pneumonia is apparent. E of FIG. 4 is an AA-I treatment group, and shows the thickening of alveolar septa and the significantly increased exudation of inflammatory cells, and there is no significant difference compared to the histamine model group, it is indicated that AA-I treatment does not have the significant improvement effect on the histamine-induced pneumonia.

F of FIG. 4 is an AA-II group, and shows the thickening of alveolar septa and increased exudation of inflammatory cells, and there is no significant difference compared to the histamine model group, it is indicated that AA-II treatment does not have the significant improvement effect on the histamine-induced pneumonia.

### Embodiment 3: Toxicity Test

1. Test Method

SPF-grade ICR mice are used, 18-22 g, with half male and half female. The animals are sourced from Beijing Vital River Laboratory Animal Technology Co., Ltd. After 1 day of adaptive feeding, the test substance is administered.

The mice are randomly divided into 4 groups: a control group (physiological saline is administered once by gavage) (1); an AA-I group (40 mg/kg of AA-I is administered once by the gavage) (2); an AA-II group (40 mg/kg of AA-II is administered once by the gavage) (3); and an AA-Iva group (40 mg/kg of AA-IVa is administered once by the gavage) (4).

Before the animals are administered by the gavage, it is fasted for 12 h without water deprivation. After being administered by the gavage, the animal death conditions within 14 days are recorded after the gavage administration. Survived animals are dissected, and kidneys are taken for the pathological examination.

2. Result

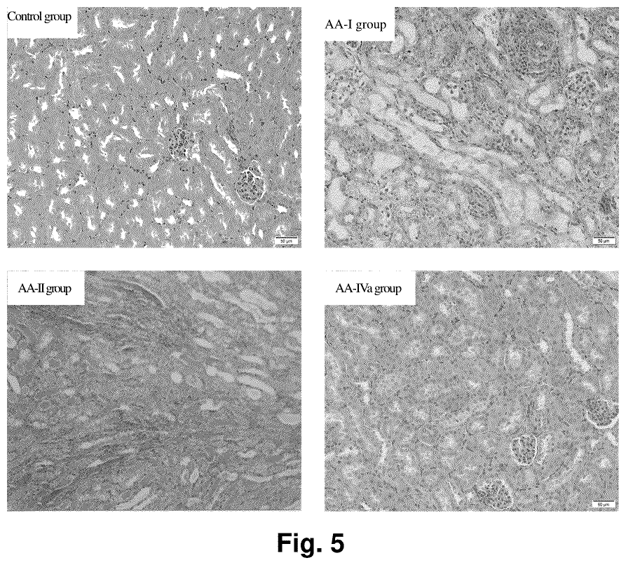

As shown in Table 3, AA-I has strong toxicity, and a single gavage administration may cause more animal deaths, 7 of 10 animals are died, and the death rate is 70%; and the renal pathological examination reveals apparent nephrotoxicity, and presents with diffuse tubular necrosis. AA-II also has apparent toxicity and may cause the animal death, and 1 of 10 animals is died (10% of the mortality rate); and the renal pathological examination reveals patchy tubular necrosis and tubular swelling and the like. AA-IVa has no apparent toxicity and no animal death; and the renal pathological examination reveals no toxic damage. The results show that AA-I and AA-II have the lethality and nephrotoxicity, while AA-IVa has no apparent toxicity and no nephrotoxicity. It is indicated that the toxicity of AA-IVa is completely different from that of AA-I and AA-II. The pathological examination results are shown in FIG. 5.

TABLE 3
Comparison of toxicity between AA-IVa and AA-I and AA-II

| | Drug | Number of | Number of |
|---|---|---|---|
| | dose | experimental | dead |
| Group | (mg/kg) | animals | animals |
| Physiological | — | 10 | 0 |
| saline | | | |
| AA-IVa | 40 | 10 | 0 |
| AA-I | 40 | 10 | 7 |
| AA- II | 40 | 10 | 1 |

It should be emphasized that the embodiments described in the present application are explanatory rather than limiting. Therefore, the present application includes but is not limited to the embodiments described in the specific implementation modes. Any other embodiments obtained by those skilled in the art based on the technical schemes of the present application also fall within the scope of protection of the present application.

1. An application of an aristolochic acid IVa in preparation of an antihistamine or a drug for treating pneumonia.

2. The application as claimed in claim 1, wherein the molecular formula of the aristolochic acid IVa is: C₁₇H₁₁NO₈, and the chemical structure formula is:

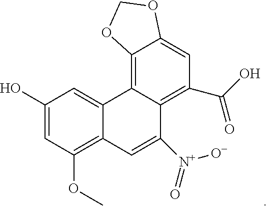 [SMILES: COc1cc(O)cc2c1cc([N+](=O)[O-])c1c(C(=O)O)cc3c(c12)OCO3]

3. The application as claimed in claim 1, wherein the use of the antihistamine comprises a use for skin allergy, eczema, dermatitis, rhinitis, or asthma.

4. The application as claimed in claim 1, wherein the pneumonia comprises pneumonia and atypical pneumonia caused by pathogens, radiation, chemicals, and allergic factors.

5. The application as claimed in claim 1, wherein the atypical pneumonia is caused by at least one of Severe Acute Respiratory Syndrome (SARS) viruses, Middle East Respiratory Syndrome (MERS) viruses, novel coronaviruses, adenoviruses, influenza viruses and legionella.

6. The application as claimed in claim 1, wherein the drug comprises the aristolochic acid IVa and a pharmaceutically acceptable carrier.

7. The application as claimed in claim 6, wherein the carrier comprises at least one of a diluent, an adhesive, an absorbent, a disintegrant, a dispersant, a wetting agent, a cosolvent, a buffering agent, and a surfactant.

8. An antihistamine, containing an aristolochic acid IVa or a medically acceptable salt thereof.

9. A drug for treating pneumonia, containing an active ingredient aristolochic acid IVa or a medically acceptable salt thereof.
